(19) United States
(12) Patent Application Publication
(10) Pub. No.: US 2026/0007838 A1
(43) Pub. Date: Jan. 8, 2026

(54) AUTOMATIC INJECTION DEVICE AND AUTOMATIC INJECTING METHOD USING THE SAME

(71) Applicants: Jongwoon Choi, Seoul (KR); DAESHIN ENTERPRISE CO., LTD., Seoul (KR)

(72) Inventors: Jongwoon Choi, Seoul (KR); Jaeil Lee, Incheon (KR)

(21) Appl. No.: 18/997,088

(22) PCT Filed: Jun. 27, 2024
(86) PCT No.: PCT/KR2024/008942

§ 371 Date: Jan. 19, 2025

(30) Foreign Application Priority Data
Dec. 1, 2023 (KR) 10-2023-0172808

(51) Int. Cl. A61M 5/42; A61M 5/20; A61M 5/32

(52) U.S. Cl. CPC A61M 5/425; A61M 5/3298; A61M 2005/2006; A61M 2205/3317; A61M 2205/3334; A61M 2205/3337; A61M 2205/3344; A61M 2210/04

(57) ABSTRACT

The present invention relates to an automatic injection device, and the automatic injection device, which administers a medicinal solution after inserting a multi-needle equipped with a plurality of injection needles into a skin at a vacuum pressure, includes: a suction motor which generates the vacuum pressure; a current detector which is provided in the suction motor to detect a current generated in the suction motor; a control unit which controls the vacuum pressure; and an air flow sensor which detects an extent in which air is sucked, wherein the vacuum pressure varies depending on the value of the current detected by the current detector and the amount of air of the air flow sensor.

### TECHNICAL FIELD

The present invention relates to an automatic injection device and an automatic injecting method using the same, and more specifically, to an automatic injecting device that adjusts a vacuum pressure by the use of a solenoid valve and a current detector which form a suction port of a fine-diameter using an injection needle, and an automatic injecting method using the same.

### BACKGROUND ART

In recent years, in current trends in which interest in a skin beauty and a skin health has increased, as the number of people who pursue skin procedures increases worldwide, interest in meso-therapy which is a skin procedure which injects medicinal solutions into the dermal and subcutaneous layers, has increased.

In general, the meso-therapy administers hyaluronic acid, which is a type of polysaccharide that can contain moisture corresponding to 300 to 1, 000 times an own weight, remove wrinkles from the skins, prevent skin aging, and improve skin elasticity. Furthermore, many people prefer the meso-therapy procedure because of an immediate administration effects and minimal pain during procedure.

Therefore, efforts are being made to develop an automatic injection device that has more excellent performance and can perform the meso-therapy more effectively.

However, despite the automatic systems that have excellent performance and are more effective are variously developed to make a great progress, there is a problem of a difficulty of inserting an injection needle evenly into the skin of people, each of which has different curvatures and elasticity.

Therefore, there is a need to develop a system that can insert an injection needle into the dermal layer and subcutaneous layer, regardless of the curvature and elasticity of each person's skin in the meso-therapy procedure.

### DISCLOSURE

### Technical Problem

The present invention aims to solve the above problems and other problems. Another object is to provide an automatic injection device and a method thereof.

### Technical Solution

In order to achieve the above and other objects, according to an aspect of the present invention, an automatic injection device, which administers a medicinal solution after inserting a multi-needle equipped with a plurality of injection needles into a skin at a vacuum pressure, includes: a suction motor which generates the vacuum pressure; a current detector which is provided in the suction motor to detect a current generated in the suction motor; and a control unit which controls the vacuum pressure, wherein the vacuum pressure may vary depending on the current detected by the current detector.

According to an aspect of the present invention, the device may include a plurality of solenoid valves which are connected to the suction motor, and block or open the inflow of outside air into the suction motor in accordance with the control of the control unit.

According to an aspect of the present invention, the control unit may control the vacuum pressure by operating the plurality of solenoid valves so that the value of the current detected by the current detector and received is maintained in accordance with the previously input value.

According to an aspect of the present invention, the device includes an air flow sensor connected to the suction motor in a direction in which air is sucked in, and when the amount of air moving to the suction motor is detected as a previously input value, the air flow sensor may administer the medicinal solution through the control unit.

According to an aspect of the present invention, the control unit may control the vacuum pressure to become an atmospheric pressure when administration of the medicinal solution is stopped.

According to an aspect of the present invention, the plurality of solenoid valves may be provided with an injection needle in a supply port to extend from the inside to the outside and bonded with an adhesive, and one side of the supply port to which the injection needle is bonded is sealed, and a suction port that penetrates by an inner diameter of the injection needle may be formed.

According to an aspect of the present invention, the suction port may each have a different diameter.

According to an aspect of the present invention, an automatic injecting method for administering a medicinal solution after inserting a multi-needle into the skin may be provided, the method including the steps of: bringing the multi-needle into close contact with the skin and operating the suction motor through the control unit; pulling up the skin by a vacuum pressure and inserting the multi-needle into the skin; receiving a value of current detected by a current detector and controlling the vacuum pressure by operating a plurality of solenoid valves selected according to the value previously input to the control unit through the control unit; injecting the medicinal solution to the skin through the control unit; and adjusting the vacuum pressure to an atmospheric pressure by operating the plurality of solenoid valves after administration of the medicinal solution is stopped.

According to an aspect of the present invention, the method may further include, after the step of controlling the vacuum pressure, a step of transmitting the amount of air detected by the air flow sensor connected to the suction motor in the direction of sucking air to the control unit.

According to an aspect of the present invention, the plurality of solenoid valves may have suction ports with different inner diameters.

### Advantageous Effects

The automatic injection device and method thereof according to the present invention are described as follows.

According to at least an embodiment of the present invention, since the current and the amount of air sucked are detected through the current detector and the air flow sensor to control the suction pressure, it is possible to apply suction pressure that is safter and does not burden the patient's skin.

According to at least an embodiment of the present invention, the suction port of the solenoid valve utilizes an injection needle and adhesive, thereby reducing the cost and time required for forming the suction port, and making it easier to manufacture a suction port with a small diameter.

According to at least an embodiment of the present invention, the suction motor can be protected from overload by maintaining a constant vacuum pressure through the solenoid valve when saturation occurs in the suction motor due to the vacuum pressure.

Further scope of applicability of the present invention will become apparent from the detailed description below. However, since various changes and modifications within the spirit and scope of the present invention will be clearly understood by those skilled in the art, it should be understood that the detailed description and specific embodiments such as the preferred embodiment of the present invention are given merely as examples.

### DESCRIPTION OF DRAWINGS

FIG. 1 is a schematic diagram of an automatic injection device according to the present invention.

FIG. 2 is a schematic diagram illustrating a solenoid valve with a suction port according to the present invention.

FIG. 3A is a schematic diagram illustrating vacuum pressure control in an automatic injection device according to the present invention.

FIG. 3B is a schematic diagram illustrating that the vacuum pressure in an automatic injection device according to the present invention is adjusted to atmospheric pressure.

FIG. 4 is a diagram illustrating the solenoid valve of the automatic injection device according to the present invention.

FIG. 5 is a flowchart of an automatic injecting method of the automatic injection device according to the present invention.

### BEST MODE

Hereinafter, the embodiments disclosed in this specification will be described in detail with reference to the accompanying drawings, and the same or similar components will be given the same reference numbers regardless of the reference numbers, and repeated descriptions will be omitted. The suffix “unit” of the components used in the following description is given or mixed in consideration of the ease of preparing the specification, and does not have a meaning or role that is distinguished from each other by itself. In addition, when describing the embodiments disclosed in this specification, if it is determined that a specific description of a related known technology makes the gist of the embodiments disclosed in this specification obscure, the detailed description will be omitted. In addition, the attached drawings are intended only to make the embodiments disclosed in this specification easily understandable, and the technical ideas disclosed in the specification are not limited by the attached drawings, and it should be understood that all modifications, equivalents, and alternatives included in the spirit and technical scope of the present invention are included.

While the terms containing ordinal numbers such as first, second, etc. may be used to describe various components, the components are not limited to the above terms. The terms are used only to distinguish one component from another.

Singular expressions include plural expressions unless the context clearly dictates otherwise.

In this application, the term such as “include” or “have” is intended to specify the presence of features, numbers, steps, operations, components, parts, or combinations thereof described in this specification, but it should not be understood that it precludes the presence or additional possibility of one or more other features, numbers, steps, operations, components, parts, or combinations thereof.

A medicinal solution 30 according to an embodiment of the present invention is injected using a syringe that can accommodate the medicinal solution 30, but is not limited thereto. The medicinal solution 30 described below includes an injector having a configuration of a hollow barrel, a piston, or a thumb rest.

An embodiment of the present invention relates to an automatic injection device 100, and more specifically, an embodiment of the automatic injection device 100 and a method thereof is disclosed in which the vacuum pressure in a suction cup 110 is adjusted using a solenoid valve 160 with a suction port 162 of a fine diameter formed thereon with an injection needle 161, and a multi-needle 111 provided inside is inserted into the skin 10 by the vacuum pressure to inject the medicinal solution 30.

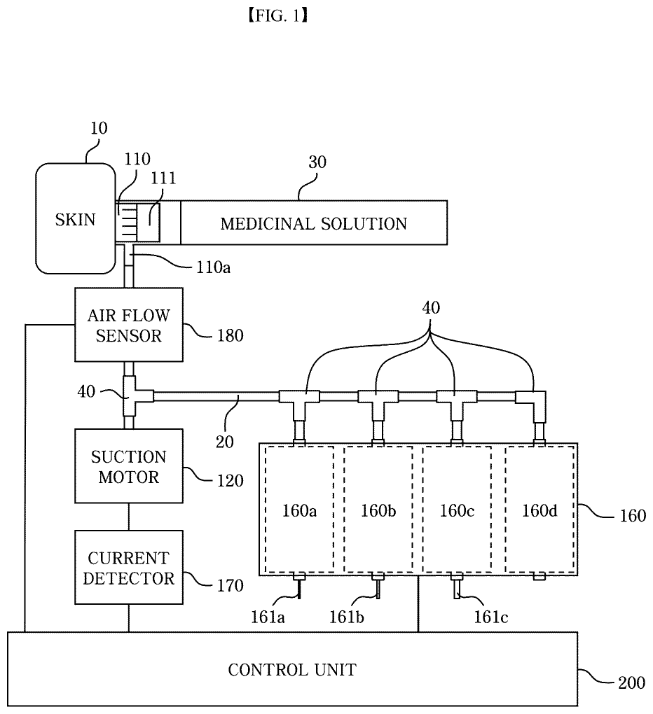

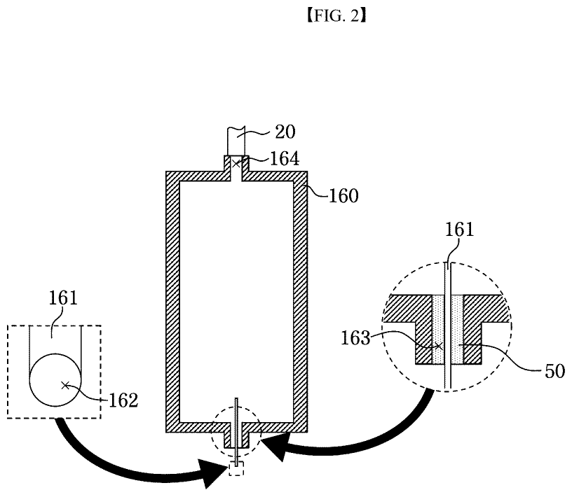

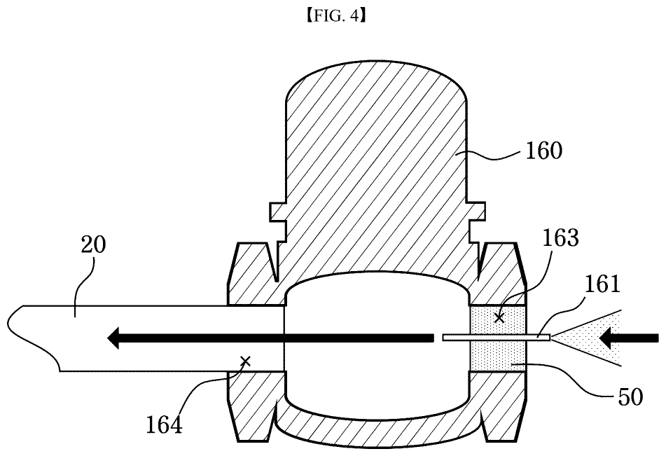

First, FIG. 1 is a schematic diagram of the automatic injection device 100 according to the present invention, FIG. 2 is a schematic diagram illustrating the solenoid valve 160 with the suction port 162 formed thereon according to the present invention, and FIG. 4 is a diagram illustrating the solenoid valve 160 of the automatic injection device 100 according to the present invention. Hereinafter, the automatic injection device 100 according to an embodiment of the present invention and a method thereof will be described with reference to FIGS. 1 and 4.

Referring to FIGS. 1, 2 and 4, the automatic injection device 100 according to an embodiment of the present invention relates to an automatic injection device 100 that administers the medicinal solution 30 after inserting a multi-needle 111 into the skin 10 with vacuum pressure. The automatic injection device 100 includes a suction motor 120 that generates the vacuum pressure, a current detector 170 that is provided in the suction motor 120 to detect a current generated in the suction motor 120, and a control unit 200 that controls the vacuum pressure, wherein the vacuum pressure has a correlation with the current detected by the current detector 170.

According to an embodiment of the present invention, the medicinal solution 30 is connected to a cylinder formed on the upper part of the suction cup 110, and the multi-needle 111, which has a plurality of needles arranged at regular intervals, is inside the suction cup 110, and is connected so that the medicinal solution 30 moves through the multi-needle 111 when the medicinal solution 30 is injected along the cylinder formed on the upper part of the suction cup 110.

In addition, the current detector 170 in an embodiment of the present invention uses a current measurement method using a Hall sensor (not shown) as a method used to detect the current flowing through the suction motor 120, but a voltage measurement method of both ends of the motor, a voltage measurement method of both ends of a resistor, and a measurement method using a current transformer can be applied.

Referring to FIG. 4, the automatic injection device includes a plurality of solenoid valves 160a, 160b, 160c, and 160d which are connected to the suction motor 120, and each have a suction port 162 formed therein so that external air is sucked in, wherein the suction port 162 is formed at the supply ports 163 of the plurality of solenoid valves 160a, 160b, 160c, and 160d, and each has a different diameter.

Here, the supply port 163 of the solenoid valve 160 (hereinafter referred to as a “valve”) is such that the supply port 163 faces the outside so that the external air can be supplied to the inside of the suction cup 110, and is connected to a hose 20 connected to a discharge port 164 from which air is transmitted through the supply port 163.

At this time, the injection needle 161 is attached to the supply port 163 of the valve 160 with an adhesive 50 to extend from inside to outside, and one side of the supply port 163 to which the injection needle 161 is attached is sealed to form a through suction port 162 having the same inner diameter as the injection needle.

In addition, since a surge may occur due to a sudden change in current with the inflow of external air, a surge suppression circuit can be designed in the suction motor 120, and a profile may be applied to the control unit to accelerate and decelerate each time the valve 160 is operated in order to prevent the suction motor 120 from damaging the skin 10 due to a sudden operation caused by a change in current.

Referring to FIG. 2, a suction port 162 having the inner diameter of the injection needle 161 is formed at the inlet of the supply port 163 to adjust the amount of suction passing through the inlet of the supply port 163. Even when forming a small hole with a dimeter of 2 mm or less, the injection needle 161 is used instead of a hole-forming device, and the material can be freely selected, which has the advantage of being cheaper and easier to manufacture.

In an embodiment of the present invention, the discharge port 164 of the solenoid valve is connected to the hose 20, and air can be introduced into the suction cup and the suction motor along the hose 20 connected through the connector.

In addition, as the injection needle 161, an injection needle that does not have a bevel at the end is used, and silicone adhesive is used as the adhesive 50, but is not limited thereto.

At this time, the hose 20 has a space formed inside so that air can move, and a hose made of PVC material is used, but elastic materials such as silicone, urethane, polyethylene (P.E), Ethylene Vinyl Acetate (EVA), Polytetra Fluoro Ethylene (PTFE) can also be adopted, and the connector 40 is attached to connect the connection port 110a of the suction cup 110, the air flow sensor 180, the suction motor 120, and the valve 160 can be connected to move air.

In an embodiment of the present invention, the valve 160 selected among the plurality of valves can be operated through the current detected by the current detector 170 to adjust the vacuum pressure.

In addition, the valve in an embodiment of the present invention is provided with four two-port direct drive valves each having the supply port 163 and the discharge port 164, but a valve having a plurality of ports similar to this or four or more valves may be provided, and is not limited thereto.

In addition, the first to fourth valves 160a, 160b, 160c, and 160d are provided with injection needles 161 having different inner diameters, the first valve 160a is provided with a first injection needle 161a, the second valve 160a is provided with a second injection needle 161b, the third valve 160c is provided with a third injection needle 161c, and the fourth valve 160d has the largest diameter to adjust the vacuum pressure acting on the suction cup 110 to atmospheric pressure during operation.

According to an embodiment of the present invention, the third injection needle 161c is formed with an area twice that of the second injection needle 161b, and the second injection needle 161b is formed with an area twice that of the first injection needle 161a.

Therefore, by utilizing the first to fourth valves 160a, 160b, 160c, and 160d, seven types of vacuum pressure can be generated, and the procedure can be carried out by varying the vacuum pressure depending on the elasticity of the patient's skin 10, and further, the degree to which the needle is inserted into the skin 10 can be controlled differently depending on the medicinal solution 30 being administered. Accordingly, there is an advantage of a wide range of versatility.

Table 1 shows the control of vacuum pressure using the first to fourth valves 160a, 160b, 160c, and 160d, “O” in the first to fourth valves 160a, 160b, 160c, and 160d indicates that the valve is closed, and “1” indicates that the valve is open, and the amount of air flowing in indicates the amount of air flowing in to the suction cup 110 through the valve 160.

TABLE 1
Solenoid valve leakage pressure adjustment step

| First | Second | Third | Fourth | Amount of air |
|---|---|---|---|---|
| valve | valve | valve | valve | flowing in |
| 0 | 0 | 0 | 0 | 0 |
| 1 | 0 | 0 | 0 | 1 |
| 0 | 1 | 0 | 0 | 2 |
| 1 | 1 | 0 | 0 | 3 |
| 0 | 0 | 1 | 0 | 4 |
| 1 | 0 | 1 | 0 | 5 |
| 0 | 1 | 1 | 0 | 6 |
| 1 | 1 | 1 | 0 | 7 |
| 0 | 0 | 0 | 1 | Atmospheric |
| | | | | pressure |

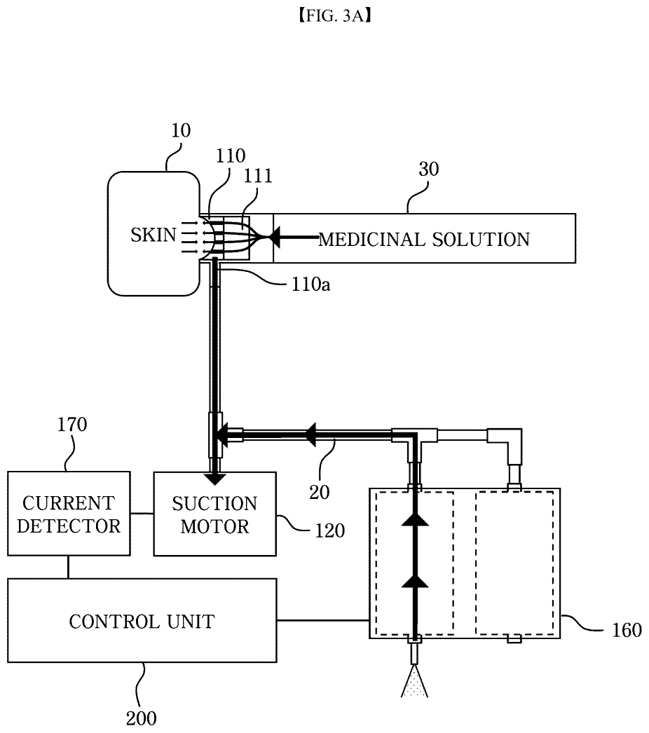

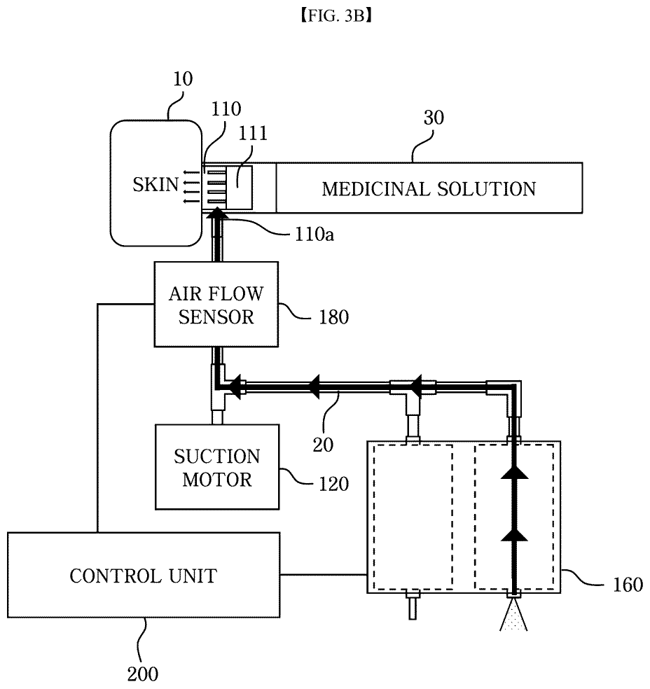

Table 1 is only for convenience of explanation only and does not limit the scope of the present invention. FIG. 3A is a schematic diagram illustrating the vacuum pressure control in the automatic injection device 100 according to the present invention, and FIG. 3B is a schematic diagram illustrating that the vacuum pressure in the automatic injection device 100 according to the present invention is adjusted to atmospheric pressure.

Referring to FIGS. 3A and 3B, the control unit 200 controls the vacuum pressure by operating the plurality of valves 160a, 160b, 160c, and 160d so that the value of the current detected and transmitted by the current detector 170 is maintained according to the previously input value, and includes an air flow sensor 180 connected to the suction motor 120 in the direction in which air is sucked in.

That is, the current detector 170 detects that the suction motor 120 generates a load due to the vacuum pressure and increases the current, and transmits this to the control unit 200, and the control unit 200 operates the valve 160 so that the value of the current is maintained at the previously input value.

Therefore, the vacuum pressure varies according to the value of the current detected by the current detector and the amount of air of the air flow sensor.

Thereafter, when the air flow sensor 180 detects the amount of air moving to the suction motor 120 as the previously input value, it administers the medicinal solution 30 to the skin 10 through the control unit 200, and when administration of the medicinal solution 30 stops, the plurality of valves 160a, 160b, 160c, and 160d can be operated through the control unit 200 to adjust the vacuum pressure to atmospheric pressure.

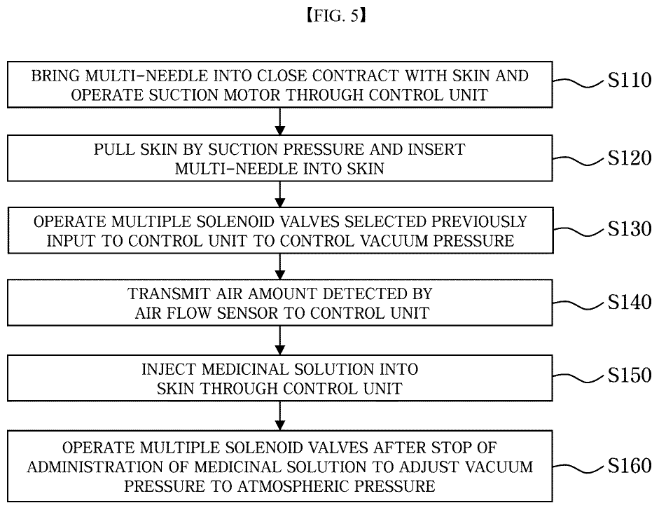

FIG. 5 is a flowchart of an automatic injecting method according to the present invention, and hereinafter, the automatic injecting method according to an embodiment of the present invention will be described with reference to FIG. 5.

Referring to FIG. 5, another embodiment of the present invention relates to an automatic injecting method for administering a medicinal solution 30 after inserting a multi-needle 111 into the skin 10 with vacuum pressure. The automatic injecting method includes a step (S110) of bringing the multi-needle 111 into close contact with the skin 10 and operating the suction motor 120 through the control unit 200, a step (S120) of pulling up the skin 10 by a vacuum pressure and inserting the multi-needle 111 into the skin 10, a step (S130) of receiving a value of current detected by a current detector 170 and controlling the vacuum pressure by operating a plurality of valves 160 selected according to the value previously input to the control unit 200 through the control unit 200, a step (S150) of injecting the medicinal solution 30 to the skin 10 through the control unit 200, and a step (S160) of adjusting the vacuum pressure to an atmospheric pressure by operating the plurality of valves 160 after administration of the medicinal solution 30 is stopped.

At this time, the plurality of valves 160 each have a suction port with a different inner diameter.

In addition, in the step (S110) of operating the suction motor, the multi-needle 111 is brought into close contact with the skin 10, and the suction motor 120 is operated through the control unit 200.

In step (S120) of inserting the multi-needle 111 into the skin 10, the skin 10 is pulled by the vacuum pressure, and the multi-needle 111 is inserted into the skin 10.

In an embodiment of the present invention, the multi-needle 111 remains fixed, and the skin 10 is pulled by the vacuum pressure, and the multi-needle 111 is inserted into the skin 10.

Then, in the step (S130) of controlling the vacuum pressure, the control unit 200 receives the value of the current detected by the current detector 170 and controls the vacuum pressure by operating a plurality of valves 160 selected according to the value previously input to the control unit 200.

In an embodiment of the present invention, the current detector 170 detects that the suction motor 120 is loaded by the vacuum pressure and the current increases, and transmits the detection result to the control unit 200, the air flowing in from the control unit 200 is input as “A1” to operate the first valve 160a, and the value of current detected so that the value of the current generated in the suction motor 120 corresponds to the flowing in air “A1” preset by the control unit 200 is maintained.

Next, step (S140) is further included in which the amount of air detected by the air flow sensor 180 connected to the suction motor 200 in the direction in which air is sucked in is transmitted to the control unit 200. The air flow sensor 180 is provided between the skin 10 and the suction motor 120 in which vacuum pressure is generated, and when the amount of air corresponding to the value previously input to the control unit 200 is maintained, the amount of detected air is transmitted to the control unit 200.

In an embodiment of the present invention, the air flow sensor 180 detects the amount of air sucked from the suction motor 120 along with the inflow air “A1” input to the control unit 200, and when the variation in the amount of air corresponding to the inflow air “A1” input to the control unit 200 is maintained constant, the air flow sensor 180 transmits a signal to the control unit 200.

Therefore, in step (S150) of injecting the medicinal solution 30 into the skin 10, the medicinal solution 30 is injected into the skin 10 through the signal transmitted from the air flow sensor 180 to the control unit 200.

Next, in step (S160) of adjusting the vacuum pressure to the atmospheric pressure, the plurality of valves 160 are operated to adjust the vacuum pressure to the atmospheric pressure after the administration of the medicinal solution 30 is stopped.

In an embodiment of the present invention, 0.1 ml of medicinal solution 30 is administered, and the control unit 200 operates the fourth valve 160d to adjust the vacuum pressure to the atmospheric pressure, and the multi-needle 111 is pulled out of the skin 10.

The above detailed description should not be construed as limiting in all respects, but should be regarded as illustrative. The scope of the present invention should be determined by a reasonable interpretation of the appended claims, and all modifications within the scope of the equivalents of the present invention are included in the scope of the present invention.

### INDUSTRIAL AVAILABILITY

One embodiment of the present invention can be used in an automatic injection device that adjusts vacuum pressure using a current detector and a plurality of solenoid valves, and an automatic injecting method using the same.

1. An automatic injection device which administers a medicinal solution after inserting a multi-needle equipped with a plurality of injection needles into a skin at a vacuum pressure, the automatic injection device comprising:
a suction motor which generates the vacuum pressure;
a current detector which is provided in the suction motor to detect a current generated in the suction motor; and
a control unit which controls the vacuum pressure,
wherein the vacuum pressure varies depending on the current detected by the current detector
an air flow sensor connected to the suction motor in a direction in which air is sucked,
wherein the air flow sensor administers the medicinal solution through the control unit when an amount of air moving to the suction motor is detected as a previously input value.

2. The automatic injection device according to claim 1, further comprising:
a plurality of solenoid valves which are connected to the suction motor, and block or open the inflow of outside air into the suction motor in accordance with the control of the control unit.

3. The automatic injection device according to claim 2, wherein the control unit controls the vacuum pressure by operating the plurality of solenoid valves so that the value of current detected by the current detector and received is maintained in accordance with the previously input value.

4. (canceled)

5. The automatic injection device according to claim 1, wherein the control unit controls the vacuum pressure to become an atmospheric pressure when administration of the medicinal solution is stopped.

6. The automatic injection device according to claim 2, wherein the plurality of solenoid valves are provided with an injection needle in a supply port to extend from the inside to the outside and bonded with an adhesive, and one side of the supply port to which the injection needle is bonded is sealed, and a suction port that penetrates by an inner diameter of the injection needle is formed.

7. The automatic injection device according to claim 6, wherein the suction ports each have a different diameter.

8-10. (canceled)
